(19) United States
(12) Patent Application Publication
(10) Pub. No.: US 2010/0291012 A1
(43) Pub. Date: Nov. 18, 2010

(54) ADMINISTRATION OF AMOROLFINE BY IONTOPHORESIS FOR TREATING NAIL DISEASES

(75) Inventors: Richard GUY, Bath (GB); Begona Delgado, Bath (GB)

Correspondence Address: BUCHANAN, INGERSOLL & ROONEY PC, POST OFFICE BOX 1404, ALEXANDRIA, VA, 22313-1404, US

(73) Assignee: GALDERMA S.A., Cham (CH)

(21) Appl. No.: 12/783,048

(22) Filed: May 19, 2010

Related U.S. Application Data
(63) Continuation of application No. PCT/EP2008/065614, filed on Nov. 14, 2008.

(51) Int. Cl. A61K 8/18

(52) U.S. Cl. 424/61

(57) ABSTRACT

A regime or regimen for the treatment of a nail disease state includes topically applying onto the infected nail(s) of an individual a thus effective amount of a solution of amorolfine in ionized form and concomitantly applying thereto an iontophoretic current of such intensity as to deliver the amorolfine into and/or through the infected nail(s).

### CROSS-REFERENCE TO PROVISIONAL/PCT APPLICATIONS

This application claims priority under 35 U.S.C. §120 of U.S. Provisional Application No. 60/996,510, filed Nov. 20, 2007, and is a continuation of PCT/EP 20087/065614, filed Nov. 14, 2008 and designating the United States (published in the English language on May 28, 2009 as WO 2009/065787 A1), each hereby expressly incorporated by reference in its entirety and each assigned to the assignee hereof.

### BACKGROUND OF THE INVENTION

1. Technical Field of the Invention

The present invention relates to the treatment of nail diseases, in particular of onychomycosis and nail psoriasis.

The present invention relates more particularly to the combined administration of a composition comprising amorolfine and of an iontophoretic current, in the treatment of nail diseases, in particular of onychomycosis and of nail psoriasis.

2. Description of Background and/or Related and/or Prior Art

Onychomycosis is a fungal nail infection which is reflected by nails that are opaque, white, thick, friable and fragile. It generally affects more than just one nail. Onychomycosis affects 2 to 13% of the population and increases to approximately 15-20% in individuals from the ages of 40 to 60.

The treatments for onychomycosis commonly employed can be divided into three categories:

systematic treatments with antifungals;

surgical procedures to completely or partly remove the infected nail, followed by a topical treatment for the exposed tissues; or else

topical applications of creams, lotions, gels or solutions to the infected nail.

These various approaches have many drawbacks.

The systemic (oral) administration of antifungal agents for the treatment of onychomycosis elicits a therapeutic effect only in the long term. For example, oral treatment with the antifungal compound ketoconazol typically requires the administration of 200 to 400 mg per day for six months before a significant therapeutic benefit is obtained. However, these antifungal agents may, in the long term, cause not insignificant adverse side effects.

Surgical removal of the nail also comprises a certain number of drawbacks, including particularly pain and discomfort associated with the surgical procedure and also the unattractive appearance of the nail.

In the case of customary topical treatments using creams, lotions, gels or solutions, the diffusion of the active ingredient through the surface of the nail is very small and the duration of the treatment is particularly long. Furthermore, these topical dosage forms do not make it possible to keep the active ingredient in contact with the nail for a sustained period and bandages therefore have to be used.

Another known topical form is nail lacquer (Murdan et al., International Journal of Pharmaceutics, 236 (2002), 1-26). Loceryl® is one example of a lacquer composed of amorolfine (5%), of Eudragit RL 100, of glycerol triacetate, of butyl acetate, of ethyl acetate and of ethanol. The lacquer is applied to the horny plate of the nail and dried for a few minutes so as to evaporate off the solvents and leave a waterproof film of polymer on the surface of the nail. The active ingredient is subsequently released from the film and diffuses through the horny plate of the nail.

Loceryl® is applied one or two times a week, for six months for the nails of the hands and nine to twelve months for the nails of the feet. The duration of treatment depends essentially on the intensity, the localization of the infection and the nail surface affected.

Treatment of the nails with a lacquer thus proves to be relatively restricting since it is repetitive, requires maintenance of the nail before each application and demands particular attention in order to avoid any contamination of the unaffected nails. Another topical form proposed is a patch containing an antifungal agent, in particular a patch containing amorolfine (WO 2005/092299), the effectiveness of which remains conditional on good penetration of the antifungal agent through the nail.

The problem shared by all the topical antifungal agents used to treat onychomycosis, and the reason for the poor effectiveness thereof, is the very weak penetration of the active ingredients through the nail in order to reach the reservoir of the infectious agent, i.e., the nail bed.

With regard to nail psoriasis, it is an inflammatory disease which can affect the matrix, the body or the bed of the nail or else the skin at the base of the nail. The damage caused to the nail may be more or less severe and may be as bad as loss of the latter. Currently, the methods for treating nail psoriasis comprise topical application of corticosteroids or of retinoids, or else local administration of glucocorticoids by local injection. However, these treatment methods remain relatively ineffective.

### SUMMARY OF THE INVENTION

The present invention features a solution to the problems related to the techniques of the prior art for treating nail diseases, by combining, under specific conditions, application, to the nail, of a composition comprising amorolfine and application of an iontophoretic current.

Iontophoresis is a method of administering medicaments through a biological membrane (typically the skin) which involves the use of an external electric field. This electric field transports the ionized molecules through the biological membrane.

The medical use of iontophoresis has been described for the treatment of various conditions, in particular cutaneous, ocular and ungual conditions (cf. WO 2005/04980 and US 2003/0144625).

However, no publication has to date described the effective combined use of a composition comprising amorolfine or a salt of amorolfine and of an iontophoretic current, in the treatment of a nail disease in a patient requiring such a treatment.

The term “salt of amorolfine” means a salt of amorolfine with a pharmaceutically acceptable acid thereof, such as, for example, an hydrochloric acid salt, so that amorolfine is in ionized form

This invention recommends maintaining the specific conditions for the effectiveness of such a combination.

The present application describes more particularly the administration of a composition comprising 1% to 10%, preferably 1% to 5%, by weight of amorolfine, preferably in ionized form in an aqueous-alcoholic solution, at a pH of from 4 to 6, formulated as a medicament for use in the treatment of a nail disease, said composition being applied to the nail in combination with an iontophoretic current, the intensity of which, which is preferably constant during application of the current, ranges from 0.01 to 10 mA/cm², preferably from 0.01 to 5 mA/cm², and even more preferably from 0.01 to 4, 3 or 2 mA/cm², for a period of time, preferably of from 0 to 24 hours, which is sufficient to enable the amorolfine to pass into or through the nail.

The iontophoretic current is used to promote the penetration, into and/or through the nail, of a therapeutically active agent, in this case amorolfine.

According to one preferred embodiment of the invention, the amorolfine is administered in ionized form, for example in the form of amorolfine hydrochloride (amorolfine HCl).

Specific preferred details of the invention are set forth in the detailed description which follows.

### BRIEF DESCRIPTION OF THE DRAWINGS

FIG. 1 indicates the cumulative delivery (nmol) of amorolfine to a receptor chamber;

FIGS. 2 and 3 indicate the cumulative delivery (nmol) of amorolfine to a receptor chamber post-iontophoresis; and

FIG. 4 shows the amount of amorolfine recovered in the nail (nmol/mg).

### DETAILED DESCRIPTION OF BEST MODE AND SPECIFIC/PREFERRED EMBODIMENTS OF THE INVENTION

### Iontophoresis

Iontophoresis employs an electric current to enable the diffusion of an ionized molecule through a biological membrane.

Under the influence of the electric current, the permeability of the biological membrane is increased, thereby promoting the passage of molecules into the cells of the biological membrane. The electric field subsequently acts on the movement of the ionized molecules through this membrane. This technique has the advantage, compared with a conventional topical application, of increasing the penetration of the active ingredient proportionally to the intensity of current applied and to the application time.

One aspect of the present invention is a device which facilitates the delivery of an active ingredient, which is very simple to use and which is capable of targeting the nail to be treated while at the same time being noninvasive. The known iontophoresis devices meet these requirements.

Any type of iontophoresis device may be suitable for the use according to the invention. Such devices have been described in the literature. According to one specific embodiment, it is possible to take advantage, in the present invention, of the transducer described in WO 2005/04980.

Such devices can be readily adapted by those skilled in the art to the use as described in the present invention, for example according to the individual to be treated, to the nature of said individual's pathology, to the number of nails affected and to the location of said nails (hand or foot).

Such a device comprises, at least, an electric current generator, an active electrode, i.e., the electrode placed on the diseased nail, and a return electrode.

The electrodes of an iontophoresis device in the context of the invention may, for example, be made in any size and shape suitable for the required use, such as circular, oval, rectangular or square shapes.

Preferably, the active electrode will have a shape best suited to the shape of the nail, i.e., of oval type. The preferred size of the active electrode according to the invention is from 0.1 cm² to 4 cm², preferably from 1 cm² to 2 cm², and is chosen according to the location of the zone to be treated.

The return electrode may be placed, for example, under the finger of the diseased nail, on the skin of the arm or of the leg, at a distance from the nail, or on the nail to be treated itself. Its shape will depend on where it is placed and its surface area will depend on the intensity of the current applied, the surface area increasing with the intensity, so as not to cause any pain (related to the passing of the current) in the individual treated.

Typically, for an active electrode of which the surface area, applied to the nail, is approximately 1 cm², and for a current with an intensity of from 0.01 to 5 mA/cm² approximately, a return electrode of which the surface area, applied to the skin, is from 1 to 10 cm² approximately will be employed.

As demonstrated by the present invention, the application of the iontophoresis technique facilitates, under specific conditions described herein, the penetration into and/or through the nail of the therapeutically active agent, optionally in combination with other active ingredients. Moreover, it allows its gradual release through the nail.

According to the present invention, the iontophoretic current is advantageously delivered after application, to the nail, of the composition comprising the therapeutic agent (described below).

The specific conditions for carrying out the iontophoresis which make it possible to effectively deliver the amorolfine into the diseased nail or through the diseased nail have now been demonstrated.

The application of the electric current may be continuous or sequential and the intensity thereof, which is preferably constant at the time of application of the current, varies, as explained above, according to the surface area of the electrodes used, and therefore within a range of from approximately 0.01 mA/cm² and approximately 10 mA/cm². The intensity is preferably constant for a given type of pair of electrodes. Advantageously, this intensity ranges from approximately 0.01 to approximately 5 mA/cm², preferably from approximately 0.5 to approximately 3 mA/cm², even more preferably from approximately 0.5 to approximately 1 mA/cm².

The current is applied for a period of time sufficient to enable the active agent(s) to pass into or through the nail, preferably to the nail bed, the reservoir of the infectious agent. This period of time ranges from approximately 1 hour to approximately 24 hours, preferably from approximately 1 hour to approximately 12 hours when the application of the current is continuous.

According to one preferred embodiment of the invention, the iontophoretic current is applied sequentially. The current is, for example, applied in cycles. The term “in cycles” means a period during which a current is applied, followed by a period during which no current is applied. The cycles are of 1 to 12 hours approximately, preferably 1 to 6 hours, at a rate of 1 to 4 cycles, preferably 2 cycles, per 24 hours.

The iontophoretic current may also, according to another embodiment of the invention, be applied in pulses, it being possible for the duration of each pulse to range from 1 second to 60 minutes approximately, it being possible for the period from each pulse to range from 1 second to 60 minutes approximately, and it being possible for the pulsed treatment to be continued for 1 to 12 hours approximately.

It has in fact now been noted, surprisingly, that such a sequential or pulsed administration facilitates, at each interruption of the current, the release, on the nail bed, of the active ingredient that has penetrated into the nail during the application of the current.

The conditions described above may be readily adapted by one skilled in the art according to the disease to be treated, the progression of this disease and the thickness of the nail.

Nail Treatment:

In the present invention, the iontophoresis is employed in combination with the application of a therapeutically active agent to the nail. This is because the iontophoresis exerts a pro-penetrating effect into the nail. The expression “pro-penetrating effect into the nail” means an effect of promoting the penetration of a molecule into or through the nail.

The invention thus relates to the formulation of a composition comprising a therapeutically active agent, for example amorolfine, preferably in ionized form, for example in the form of amorolfine hydrochloride, into a medicament for use in the treatment of a nail disease, said composition being applied to the nail in combination with an iontophoretic current, for a period of time sufficient to enable said agent to pass into and/or through the nail.

The term “treatment” means preventive, palliative or curative treatments, allowing stabilization or complete curing of the disease.

Preferably, the invention features the formulation, as described above for example, into a medicament for use in the treatment of onychomycosis or of nail psoriasis.

All types of onychomycosis are targeted, namely, in particular, distal subungual onychomycosis, proximal white subungual onychomycosis, white superficial onychomycosis and onychomycosis induced by a strain of the Candida genus. All types of nail psoriasis are also targeted.

The therapeutically active agents may be applied before or after application of the iontophoretic current to the nail. Thus, in one preferred embodiment, the user applies the active agent in one of the topical forms described below and then applies an iontophoretic current to the nail coated with the active agent. In an alternative embodiment, the iontophoretic current is first applied to the nail completely or partially coated with a cream, a gel, a lotion or a solution devoid of therapeutically active agent in order to prepare the nail for the penetration of the therapeutically active agent used. The latter is subsequently applied to the nail thus prepared. The iontophoretic current may or may not then be again applied to the nail, the latter being partially or completely coated with said therapeutically active agent.

In one particular embodiment of the invention, the therapeutically active agent is present in the form of a free acid or base.

The term “therapeutically active agent” means a molecule which is effective against the nail disease to be treated.

In one particular embodiment, the therapeutically active agent is an antifungal agent that is effective in the treatment of onychomycosis.

Any antifungal agent known to those skilled in the art to be effective in the treatment of onychomycosis or of nail psoriasis may be used in combination with the application of an iontophoretic current to the nail. Preferably, the antifungal agent is amorolfine, preferably amorolfine hydrochloride (or amorolfine HCl) or a derivative thereof. The use of an iontophoretic current to promote the penetration into or through the nail of medicaments which are not at the current time used in the treatment of onychomycosis owing to their weak penetration into the nail, may also be envisaged.

The term “amorolfine” means the amorolfine base. The term “amorolfine derivatives” means in particular the ionized forms thereof, in particular the pharmaceutically acceptable salts thereof, and more particularly the hydrochloride derivative thereof (amorolfine HCl).

The term “pharmaceutically acceptable salts” means salts that are compatible with the integuments, and preferably with the skin and/or the mucous membranes.

Amorolfine HCl or amorolfine hydrochloride is a hydrochlorinated derivative of amorolfine and denotes the hydrochloride of the acid cis-4-[3-[4-(1,1-dimethylpropyl)phenyl]-2-methylpropyl]-2,6-dimethylmorpholine represented by formula (1):

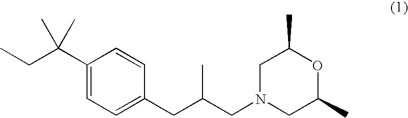

This chemical compound exerts a fungistatic and fungicidal activity by inhibiting the synthesis of the cell membrane sterols of fungi such as yeasts, dermatophytes, moulds and dematiaceous fungi (black fungi).

The compositions according to the present invention preferably comprise 1% to 10%, even more preferably 1% to 5%, by weight of amorolfine, preferably in ionized form, for example in the form of amorolfine hydrochloride, as therapeutically active antifungal agent.

The amorolfine or a derivative thereof may be present in combination with another active substance suitable for transungual application, having biological or pharmacological, and in particular antibiotic or antifungal, properties, via topical application to the area of the nail to be treated.

Advantageously, the therapeutically active agent may be applied to the nail in combination with an agent for promoting absorption into the nail. The term “promoting absorption into the nail” means pharmaceutically acceptable chemical compounds capable of increasing the permeability of a biological membrane such as the skin or the nail with respect to a therapeutically active agent such as amorolfine or derivatives thereof, so as to increase the kinetics for penetration of the therapeutically active agent or derivatives thereof through the membrane.

These penetration kinetics can be measured using techniques well known to those skilled in the art. Thus, use may be made of a cellular diffusion apparatus such as the Franz cells described by Merrit et al. (Diffusion Apparatus for Skin Penetration, J. Controlled Release, 1984, 1, 161-162) or else the method described in Walters et al. (Fate of substances delivered to follicles, ducts and nails, ASCC, 2005) or in Gupchup et al. (Structural characteristics and permeability properties of the human nail: a review, J. Cosmet. Sci., 1999, 50, 363-385). Another method is described in WO 2005/011565.

The absorption promoters are well known in the prior art and may include, in particular, α-hydroxy acids, esters of fatty acids and amides thereof, fatty alcohols, fatty acids and glycerol esters, in particular 2-(2-ethoxyethoxy)ethanol, glyceryl monolaurate, propylene glycol, polyethylene glycols, polyglycosylated glycerides, unsaturated polyglycols (Labrafil M1944CS®, Gattefosse), saturated polyglycerides (Labrasol, Gattefosse), Labrafac HydroWL 1219® (Gattefosse), decylmethyl sulfoxide, pyrrolidones, salicylic acid, lactic acid, isopropyl myristate, dimenthylformamide, dimethylacetamide, sodium dodecyl sulfate, phospholipids, Transcutol® (Gattefosse), and mixtures of oleic acid and 2-(2-ethoxyethoxy)ethanol and of oleic acid and Labrafil®, these oleic acid mixtures preferably being in a ratio of approximately 1:1. Enzymatic compounds, such as proteolytic enzymes which facilitate the penetration of active ingredients through keratinous tissues or through the nail, may also be used as absorption promoters. By way of non-limiting examples of fatty acids that can be used according to the invention, exemplary are capric acid, lauric acid, myristic acid, palmitic acid, stearic acid, arachidic acid, behenic acid, lignoceric acid, myristoleic acid, palmitoleic acid, petroselinic acid, oleic acid, linoleic acid and linolenic acid.

Other known absorption promoters function by means of hydrolysis, keratolysis, denaturation or another equivalent mechanism which destroys the nail or the membrane. Examples of absorption promoters that function in this manner are urea, amino acids comprising sulfydryl groups, alkyl sulfoxides, and any equivalent compound which functions by destroying or denaturing the nail and/or the membrane thus enabling the pharmaceutical compound to penetrate the deep layers of the membrane.

Examples of absorption promoters include urea, exaltolide, N-acetylcysteine and lactic acid, or a mixture thereof, urea combined with lactic acid or with N-acetylcysteine, and exaltolide alone, being particularly preferred.

The amount of each absorption promoter may be determined by those skilled in the art according, in particular, to the administration form of the therapeutically active agent, to the appearance of the nail and/or to the progression of the disease to be treated.

The compositions according to the present invention optionally also comprise one or more solvents.

A solvent is a substance with which the other ingredients in the composition mix or dissolve or are suspended, softened and/or liquefied such that such a composition may be applied topically to a surface such as a nail. The solvent(s) should be physiologically acceptable.

Suitable physiologically acceptable solvents include water, hydrocarbons, halogenated hydrocarbons, alcohols, ethers, ketones and esters that are of use in beauty products, such as acetic esters of monohydric alcohols (ethyl and butyl acetates, for example), optionally mixed with aromatic hydrocarbons, such as toluene, and/or alcohols such as ethanol or isopropanol.

The choice of solvent is essentially determined by its ability to solubilize the active agent(s).

As regards amorolfine or derivatives thereof, an aqueous-alcoholic solution, typically comprising ethanol, is preferably used.

The pH of this solution advantageously ranges from 3 to 6 for amorolfine and derivatives thereof.

The use of a cosolvent or of a mixture of cosolvents may also prove to be beneficial. Isopropanol is particularly useful as a cosolvent for solubilizing the absorption promoter pair urea/lactic acid.

Of course, one skilled in the art may, in the light of their general knowledge, determine the solvents and cosolvents which are most suitable for the working conditions and the required degree of solubilization of the constituents.

The therapeutically active agent, optionally combined with an absorption promoter, is preferably applied in the form of a lotion, a cream, a gel, a patch, a solution or any other acceptable form.

In one particular embodiment, the therapeutically active agent may be present in the form of a free acid or base.

The composition may also advantageously comprise at least one salt or any other agent carrying an electrical charge, for example in the form of a buffer solution. This may be the case, for example, when the active agent is not in the form of a salt.

The therapeutically active agent is present, in the compositions according to the present invention, in an amount that is effective for treating the nail pathology from which the individual is suffering.

Typically, when the pathology to be treated is onychomycosis, the agent is an antifungal agent such as amorolfine or a derivative thereof. The latter is preferably present, in the composition, in an amount of from approximately 1% to approximately 10%, preferably from approximately 1% to approximately 5%, even more preferably from approximately 1% to approximately 3% by weight of said composition. The weight of composition comprises the whole of the volatile and non-volatile ingredients possibly present in said composition (the amorolfine being included in this whole).

In one particular embodiment, this invention thus features the formulation of a composition preferably comprising 1% to 5% by weight of amorolfine, preferably in the form of amorolfine hydrochloride, preferably in an aqueous-alcoholic solution, at a pH advantageously ranging from 3 to 6, into a medicament useful for the treatment of a nail disease, in particular of onychomycosis or of nail psoriasis, said composition being applied to the nail in combination with an iontophoretic current, the intensity of which is preferably constant and preferably from 0.01 to 5 mA/cm², for a period of time which is sufficient to enable the amorolfine to pass into or through the nail.

In yet another embodiment of the present invention, the composition comprises approximately 1% by weight of amorolfine hydrochloride in a Tris/ethanol solution at a pH of from 3 to 5, and the intensity of the iontophoretic current applied is approximately 1 mA/cm².

In one particular embodiment, the nail is prepared before the application of the iontophoretic current or of the pharmaceutical composition containing the therapeutically active agent. For example, the nail may be preconditioned by pretreatment with an absorption promoter or by means of an occlusive patch in order to hydrate it. Such an occlusive path is described in FR-2871292.

Alternatively or in a combined manner, the nail may also be prepared by abrasion using a nail file or using a chemical composition that is abrasive for the nail, such as that described in US 2004/0197280.

The invention relates, moreover, to the kits and methods for prophylactic or therapeutic treatment associated with the uses described above.

It relates, for example, to a method for treating a nail pathology, comprising (i) the application, to the diseased nail, of a composition comprising a therapeutically active agent, for example amorolfine, preferably in the form of amorolfine hydrochloride, and (ii) the application of an iontophoretic current, for a period of time which is sufficient to enable said agent to pass into and/or through the nail.

One particular embodiment of the invention thus relates to a method for treating onychomycosis, comprising (i) the application, to the diseased nail, of a composition comprising 1% to 5% by weight of amorolfine in an aqueous-alcoholic solution, at a pH of from 4 to 6, and (ii) the application of an iontophoretic current, the intensity of which is preferably constant and ranges from approximately 0.01 to approximately 5 mA/cm², for a period of time which is sufficient to enable the amorolfine to pass into and/or through the nail.

The present invention also features a regime or regimen for treating onychomycosis, comprising (i) the application, to the diseased nail, of a composition comprising 1% by weight of amorolfine hydrochloride in a Tris/ethanol solution at a pH of from 4 to 5, and (ii) the application of an iontophoretic current, the intensity of which, which is preferably constant, is approximately 1 mA/cm², for a period of time which is sufficient to enable the amorolfine to pass into and/or through the nail.

It should be noted that individuals who suffer from the nail pathologies mentioned above, in particular from onychomycosis or from nail psoriasis, can apply compositions according to the present invention to healthy or non-infected nails preventively, in order to prevent the appearance or spreading of the pathology.

Elderly patients and immunodepressed patients are also individuals to whom the prophylactic therapy according to the present invention may apply.

In order to further illustrate the present invention and the advantages thereof, the following specific examples are given, it being understood that same are intended only as illustrative and in nowise limitative. In said examples to follow, all parts and percentages are given by weight, unless otherwise indicated.

### EXPERIMENTAL SECTION

### Experimental Conditions

### Experiments 2; 3; 4 and 5

Donor Solution

The donor solution in experiment 2 consisted of: 0.36% (10 mM) amorolfine HCl in an aqueous solution containing 50 mM NaCl and 0.4% Tween 80. The experiment was performed at pH 3.9 without further adjustment.

The donor solution in experiment 3 consisted of: 0.36% (10 mM) amorolfine HCl in an 40:60 (w:w) Tris buffer:ethanol. The Tris buffer contained 50 mM Tris and 50 mM NaCl at pH 7.4. The pH of the buffer:ethanol solution was ˜7.5. After addition of the drug the pH was ˜5.5.

The donor solution in experiments 4 and 5 consisted of: 1% (28 mM) amorolfine HCl in an 40:60 (w:w) Tris buffer:ethanol. The Tris buffer contained 50 mM Tris and 50 mM NaCl at pH 7.4. The pH of the buffer:ethanol solution was ˜7.5. After addition of the drug the pH was ˜4.7.

Receptor solution: pH 7.4 PBS containing 0.25% Tween 80 (same as Expt. 2).

Experimental Set-Up:

Different diffusion cells (side-by-side and Franz) and alternative nail assemblies were considered. The set-up described below was selected for these experiments:

Diffusion cells: 15 mm diameter Franz diffusion cells were used. The “nail assemblage” was placed in from the donor and receptor chambers.

Nail device: A nail was glued from two silicone rings with an internal orifice of 5 mm diameter. Acrylic glue was used as other alternatives (silicone grease, other bioadhesive polymers) did not hold the nail “in place”, resulting either in leaks or spreading of the grease onto the nail surface.

The thickness of the nails was measured with a micrometer. Before hydration nail thickness in the periphery was measured in three places. The central part was not measured at this time to avoid any risk of breaking the nail. The nails were soaked overnight in 50 mM NaCl solution. Because of the increased flexibility of the nail after soaking, it was then possible to measure the thickness in the central part, upper part and lateral corner of the nail (Table 1):

TABLE 1

| Expt. | Nail A | Nail B | Nail C |
|---|---|---|---|
| 3 | 373 ± 27 | 315 ± 17 | 280 ± 13 |
| 4 | 143 ± 36 | 170 ± 54 | 315 ± 87 |
| 5 | 135 ± 59 | 188 ± 83 | 123 ± 40 |

Testing for leaks and iontophoretic circuit: Before starting the iontophoretic experiment, the absence of leaks was tested for each cell. The donor and receptor chambers were filled with donor (without drug) and receptor buffer, respectively, and a 0.1 mA current was passed. In the presence of leaks, the resistance of the circuit is low and the voltage needed to drive the current is very small (˜1V). A leak-free system typically resulted in much higher voltages (˜100 V). No leaks were observed in any case.

The voltages observed at the start of current passage decreased rapidly to settle at (˜30V after approximately 20 minutes.

Once the stability of the circuit with the three cells was assured, the donor compartment was filled with the drug solution and the current re-started.

The intensity of current was 0.1 mA, equivalent to 0.51 mA/cm² (area exposed=0.196 cm²).

One sample was taken after 18 hours of iontophoresis for experiment 2 and 20 hours for experiments 3 and 4. The receptor was then refilled and a passive post-iontophoretic period of 4 hours elapsed before a second sample was obtained.

After recovery of the donor solution, the anode chamber was rinsed twice with ethanol to clean residual solution from the surface of the nail. Then, the nail was cut out of the silicone ring, weighed, dissolved in Soluene®, and assayed for radioactivity.

Summary of the Experimental Conditions:

Experiment 5 is a passive control.

| | | | Current intensity |
|---|---|---|---|
| Experiment | Donor solution | [AmHCl] | & duration |
| #2 | 50 mM NaCl + | 0.36% | 0.1 mA × 18 h + |
| | 0.25% Tween 80 | (10 mM) | 0 mA × 4 h |
| | pH = 3.9 | | |
| #3 | 50 mM | 0.36% | 0.1 mA × 20 h + |
| | Tris/Ethanol | (10 mM) | 0 mA × 4 h |
| | 60:40 | | |
| | pH = ~5.5 | | |
| #4 | 50 mM | 1% | 0.1 mA × 20 h + |
| | Tris/Ethanol | (28 mM) | 0 mA × 4 h |
| | 60:40 | | |
| | pH ~4.7 | | |
| #5 | 50 mM | 1% | 0 mA × (20 + 4) h |
| | Tris/Ethanol | (28 mM) | Passive Control |
| | 60:40 | | |
| | pH ~4.7 | | |

Results:

The cumulative delivery (nmol) of Amorolfine to the receptor compartment in shown is Table 2, and FIG. 1.

TABLE 2

| Experiment | Cell A | Cell B | Cell C |
|---|---|---|---|
| # 2 (18 hr × 0.1 mA) | 1.67 | 2.38 | 34.9 |
| # 3 (20 hr × 0.1 mA) | 0.97 | 1.24 | 2.28 |
| # 4 (20 hr × 0.1 mA) | 5.91 | 3.77 | 13.9 |
| # 5 (20 hr passive) | 0.91 | 0.48 | 0.37 |

The comparison amorolfine flux (nl/hr) from the 3 experiments is given in the Table 3.

TABLE 3

| Experiment | Cell A | Cell B | Cell C |
|---|---|---|---|
| # 2 (18 hr × 0.1 mA) | 1.67 | 2.38 | 34.86 |
| # 3 (20 hr × 0.1 mA) | 0.97 | 1.24 | 2.28 |
| # 4 (20 hr × 0.1 mA) | 5.91 | 3.77 | 13.9 |
| # 5 (20 hr passive) | 0.91 | 0.48 | 0.37 |

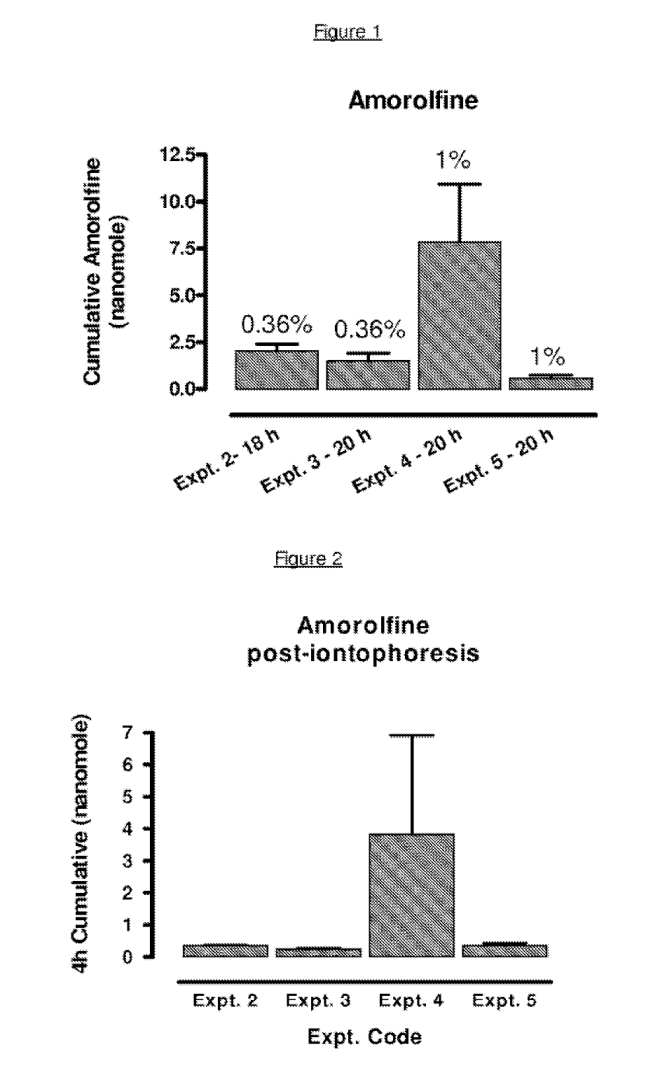

The cumulative delivery (nmol) of Amorolfine to the receptor compartment post-iontophoresis in shown is Table 4, and FIGS. 2 and 3.

TABLE 4

| Experiment | Cell A | Cell B | Cell C |
|---|---|---|---|
| # 2 (18-22 hr) | 0.34 | 0.37 | 11.2 |
| # 3 (20-24 hr) | 0.26 | 0.19 | 0.27 |
| # 4 (20-24 hr) | 7.33 | 2.61 | 1.55 |
| # 4 (20-24 hr) | 0.39 | 0.41 | 0.28 |

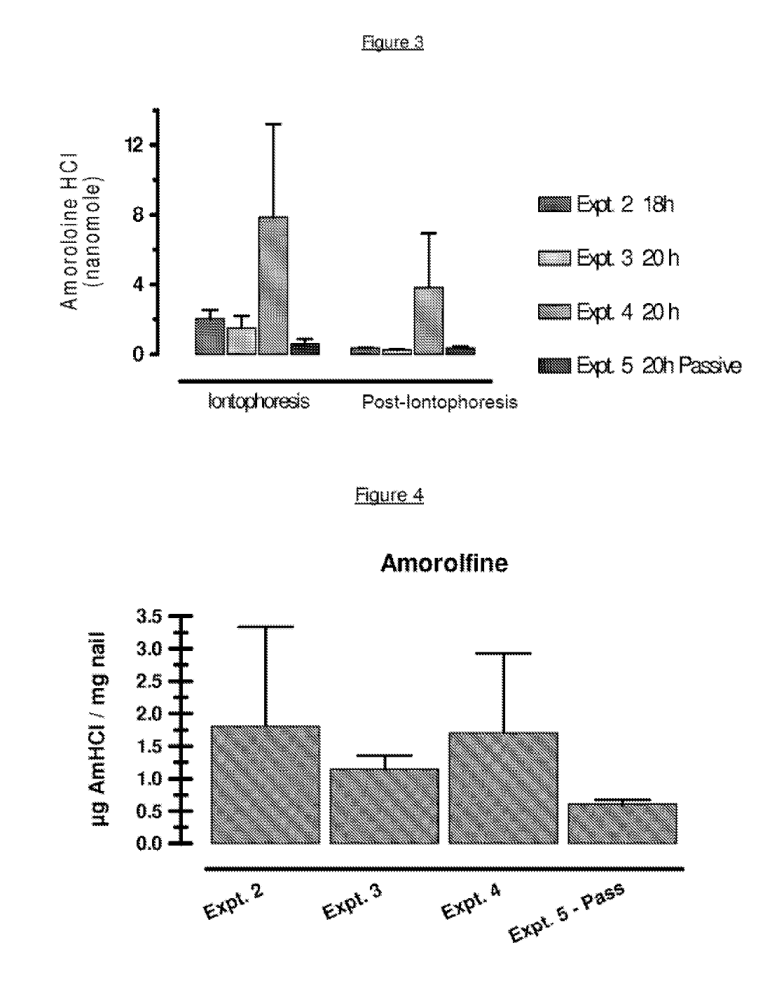

The amorolfine recovered in the nail (nmol/mg) is shown in Table 5, and FIG. 4.

TABLE 5

| Experiment | Cell A | Cell B | Cell C |
|---|---|---|---|
| # 2 (18 hr × 0.1 mA) | 10 | 2.4 | 2.9 |
| # 3 (20 hr × 0.1 mA) | 2.8 | 3.9 | 3.0 |
| # 4 (20 hr × 0.1 mA) | 8.7 | 3.8 | 2.0 |
| # 5 (20 hr passive) | 1.5 | 1.9 | 1.8 |

Summary of Amorolfine Results:

| | Receptor | Total nail | Nail |
|---|---|---|---|
| Experiment | (nmol) | (nmol) | (nmol/mg) |
| # 2 (18 hr × 0.1 mA) | 2.4 ± 0.5 | 38 ± 13 | 5.1 ± 4.3 |
| # 3 (20 hr × 0.1 mA) | 1.7 ± 0.7 | 28 ± 3.6 | 3.2 ± 0.6 |
| # 4 (20 hr × 0.1 mA) | 12 ± 4.7 | 57 ± 29 | 4.8 ± 3.5 |
| # 5 (20 hr passive) | 1.0 ± 0.3 | 18 ± 1.1 | 1.7 ± 0.2 |

The results suggest a higher flux of amorolfine in Expt. 4 as compared with Expt. 2 and 3.

Iontophoresis of Amorolfine enhances drug delivery into and trough the nail.

Each patent, patent application, publication, text and literature article/report cited or indicated herein is hereby expressly incorporated by reference in its entirety.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A regime or regimen for the treatment of a nail disease state, comprising topically applying onto the infected nail(s) of an individual a thus effective amount of a solution of amorolfine in ionized form and concomitantly applying thereto an iontophoretic current of such intensity as to deliver said amorolfine into and/or through said infected nail(s).

2. The regime or regimen as defined by claim 1, said solution comprising 1% to 5% by weight of amorolfine in ionized form in an aqueous-alcohol solution and having a pH ranging from 3 to 6.

3. The regime or regimen as defined by claim 2, the intensity of said iontophoretic current being essentially constant and ranging from about 0.01 to about 5 mA/cm².

4. The regime or regimen as defined by claim 3, said iontophoretic current being applied for up to 24 hours.

5. The regime or regimen as defined by claim 1, in which the iontophoretic current is delivered after application of said amorolfine solution to said infected nail(s).

6. The regime or regimen as defined by claim 1, in which the iontophoretic current is applied continuously for about 1 to 12 hours.

7. The regime or regimen as defined by claim 1, in which the iontophoretic current is applied sequentially.

8. The regime or regimen as defined by claim 7, in which the iontophoretic current is applied in cycles of 1 to 6 hours, at a rate of about 2 to 4 cycles per 24 hours.

9. The regime or regimen as defined by claim 1, in which the intensity of the iontophoretic current applied ranges from about 0.5 to about 3 mA/cm².

10. The regime or regimen as defined by claim 1, in which the intensity of the iontophoretic current applied ranges from about 0.5 to about 1 mA/cm².

11. The regime or regimen as defined by claim 1, in which the amorolfine solution also comprises an agent for promoting absorption into the nail.

12. The regime or regimen as defined by claim 8, in which the absorption-promoting agent comprises urea.

13. The regime or regimen as defined by claim 1, in which the ionized amorolfine comprises amorolfine hydrochloride (amorolfine HCl).

14. The regime or regimen as defined by claim 1, in which the aqueous-alcoholic amorolfine solution comprises ethanol.

15. The regime or regimen as defined by claim 1, in which the amorolfine solution comprises 1% by weight of amorolfine hydrochloride in a Tris/ethanol solution at a pH of from 3 and 5, and in which the intensity of the iontophoretic current applied is about 1 mA/cm².

16. The regime or regimen as defined by claim 1, for the treatment of onychomycosis.

17. The regime or regimen as defined by claim 1, for the treatment of nail psoriasis.
